(19) United States
(12) Patent Application Publication
(10) Pub. No.: US 2025/0326759 A1
(43) Pub. Date: Oct. 23, 2025

(54) PROCESS FOR PREPARING 1-[5-TERT-BUTYL-3-[(1-METHYLTETRAZOL-5-YL)METHYL]TRIAZOLO[4,5-D]PYRIMIDIN-7-YL]PYRROLIDIN-3-OL

(71) Applicant: Hoffmann-La Roche Inc., Little Falls, NJ (US)

(72) Inventors: Jean-Michel ADAM, Rosenau (FR); Dainis KALDRE, Loerrach (DE); Christian Oliver KAPPE, Graz (AT); Christian Steffen MOESSNER, Loerrach (DE); Michael PRIESCHL, Graz (AT); Peter SAGMEISTER, Graz (AT); Joerg Mathias SEDELMEIER, Basel (CH); Sebastian TROKOWSKI, Giebenach (CH); Jason Douglas WILLIAMS, Graz (AT)

(73) Assignee: Hoffmann-La Roche Inc., Little Falls, NJ (US)

(21) Appl. No.: 18/970,351

(22) Filed: Dec. 5, 2024

Related U.S. Application Data
(63) Continuation of application No. PCT/EP2023/064913, filed on Jun. 5, 2023.

(30) Foreign Application Priority Data
Jun. 7, 2022 (EP) 22177552.1

(51) Int. Cl. C07D 487/04; C07D 257/04; C07D 403/06

(52) U.S. Cl. CPC C07D 487/04; C07D 257/04; C07D 403/06

(57) ABSTRACT

The present invention relates to a process for the preparation of (3S)-1-[5-tert-butyl-3-[(1-methyltetrazol-5-yl)methyl]-6,7-dihydrotriazolo[4,5-d]pyrimidin-7-yl] pyrrolidin-3-ol useful as pharmaceutically active compounds.

The present invention relates to a process for the preparation of 1-[5-tert-butyl-3-[(1-methyltetrazol-5-yl)methyl] triazolo[4,5-d]pyrimidin-7-yl]pyrrolidin-3-ol useful as pharmaceutically active compounds.

1-[5-tert-butyl-3-[(1-methyltetrazol-5-yl)methyl] triazolo[4,5-d]pyrimidin-7-yl]pyrrolidin-3-ol, compound of formula (VI) as described in WO2013/068306 has shown as valuable pharmaceutical compound, as CB2 receptor agonist. The interest in CB2 receptor agonists has been steadily on the rise during the last decade (currently 30-40 patent applications/year) due to the fact that several of the early compounds have been shown to have beneficial effects in pre-clinical models for a number of human diseases including chronic pain (Beltramo, M. Mini Rev Med Chem 2009, 9 (1), 11-25), atherosclerosis (Mach, F. et al. J Neuroendocrinol 2008, 20 Suppl 1, 53-7), regulation of bone mass (Bab, I. et al. Br J Pharmacol 2008, 153 (2), 182-8), neuroinflammation (Cabral, G. A. et al. J Leukoc Biol 2005, 78 (6), 1192-7), ischemia/reperfusion injury (Pacher, P. et al. Br J Pharmacol 2008, 153 (2), 252-62), systemic fibrosis (Akhmetshina, A. et al. Arthritis Rheum 2009, 60 (4), 1129-36; Garcia-Gonzalez, E. et al. Rheumatology (Oxford) 2009, 48 (9), 1050-6), liver fibrosis (Julien, B. et al. Gastroenterology 2005, 128 (3), 742-55; Munoz-Luque, J. et al. J Pharmacol Exp Ther 2008, 324 (2), 475-83). Without wishing to be bound by theory, using azides in a flow reactor may prevent and/or reduce the build-up of explosive components which are commonly found in typical syntheses involving azides. For example, due to the lack of headspace in the tubular flow reactor, volatile HN₃ may not accumulate in any significant quantity. For example, with batch processes, there is a risk of HN₃ accumulation and condensation in the headspace, a risk of heavy metal azide deposition, a risk of personnel exposure to HN₃, and/or a requirement of a use of a restricted temperature range. In contrast, for continuous flow processes, HN₃ accumulation is minimized in the reactor (e.g., tubular reactor) due to the lack of headspace, reactor volumes are small, a variety of temperature ranges are accessible, including higher ranges as compared to batch processes, and/or the process allows for inline quenching of excess azide residues and other hazardous azide waste products.

HN₃ is a known side product, which is generally formed when azide reagents are used under acidic conditions. HN₃ is a very volatile compound (bp=37° C.) and is reported to form explosive gas phase mixtures with air and/or nitrogen in concentrations as low as 8-15% by volume. Neat HN₃ is extremely explosive, shock sensitive, and highly toxic (e.g., the recommended airborne exposure limit for hydrazoic acid is 0.11 ppm (0.3 mg/m as sodium azide) according to the National Institute for Occupational Health and Safety (NIOSH). Due to the flowing nature of the reaction and the ability to quench any unreacted azide and/or HN₃ prior to removing the reaction mixture from the flow reactor, the risk of potential explosion and exposure is greatly reduced and/or eliminated. Thus, the methods of the present invention can minimize and/or eliminate the possibility of build-up of large amounts of HN₃ in either gaseous or liquid form.

The present invention takes place in the presence of a solvent or a mixture of two or more solvents. In particular the solvent can be an organic solvent such as an ether like solvent (e.g. tetrahydrofuran, acetonitrile, diisopropyl ether, tert-butylmethyl ether or dibutyl ether, in particular acetonitrile), an alcohol solvent (e.g. methanol, n-butanol, s-butanol, tert-butanol, or ethanol, in particular t-butanol), an aliphatic hydrocarbon solvent (e.g. hexanes, heptanes or pentane), a saturated alicyclic hydrocarbon solvent (e.g. cyclohexane or cyclopentane) or aromatic solvent (e.g. toluene or t-butyl-benzene), polar aprotic solvent (e.g. acetonitrile, DMSO, sulfolane), water or a combination thereof. More particularly in some steps of the invention the solvent of particular interest are acetonitrile, while in other steps of the invention the solvent of particular interest is ethyl acetate.

A first aspect of the present invention provides a process for the preparation of compound of formula (II)

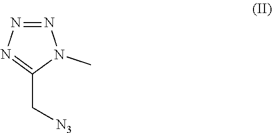

which comprises reacting compound of formula (I)

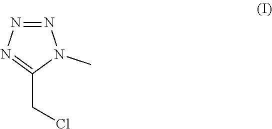

in the presence of an azide source.

A second aspect of the present invention provides a process for the preparation of compound of formula (III)

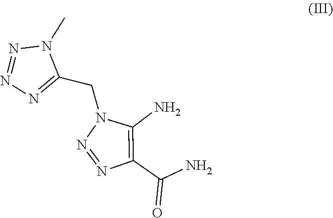 [SMILES: Cn1nnnc1Cn1nnc(C(N)=O)c1N]

which comprises reacting compound of formula (II)

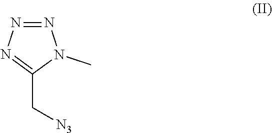

with 2-cyanoacetamide.

A third aspect of the present invention provides a process for the preparation of compound of formula (IV)

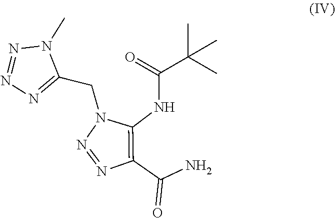 [SMILES: Cn1nnnc1Cn1nnc(C(N)=O)c1NC(=O)C(C)(C)C]

which comprises reacting compound of formula (III)

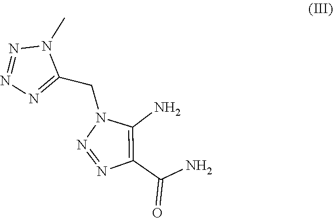 [SMILES: Cn1nnnc1Cn1nnc(C(N)=O)c1N]

with a pivaloyl source.

A forth aspect of the present invention provides a process for the preparation of compound of formula (V)

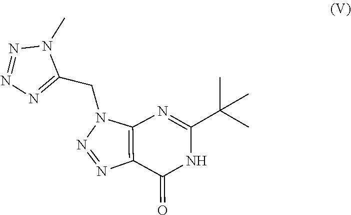 [SMILES: Cn1nnnc1Cn1nnc2c(=O)[nH]c(C(C)(C)C)nc21]

which comprises reacting compound of formula (IV)

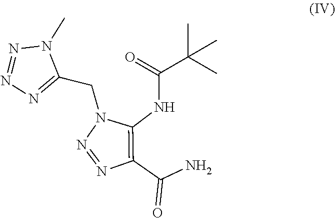 [SMILES: Cn1nnnc1Cn1nnc(C(N)=O)c1NC(=O)C(C)(C)C]

with a base.

A fifth aspect of the present invention provides a process for the preparation of compound of formula (VI)

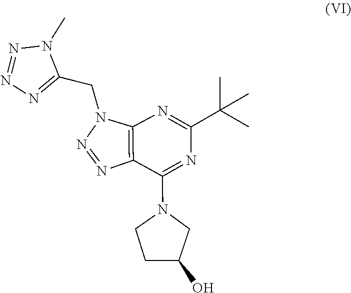 [SMILES: Cn1nnnc1Cn1nnc2c(N3CC[C@H](O)C3)nc(C(C)(C)C)nc21]

which comprises reacting compound of formula (V)

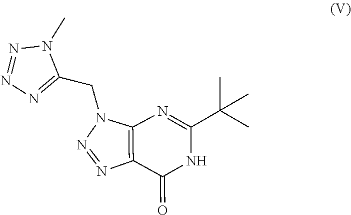 [SMILES: Cn1nnnc1Cn1nnc2c(=O)[nH]c(C(C)(C)C)nc21]

with compound of formula (VII)

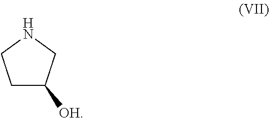

### BRIEF DESCRIPTION OF THE FIGURES

FIGS. 1A and 1B illustrates the flow process of synthesis from the compound of formula I to the compound of formula III. T¹ and T² refer to temperature in ° C. According to the present invention, T¹ can be between 50° C. and 150° C., particularly between 70° C. and 120° C., more particularly between 90° C. and 120° C., T² can be between 0° C. and 70° C., in particular between 30° C. and 50° C., more particularly at 40° C.

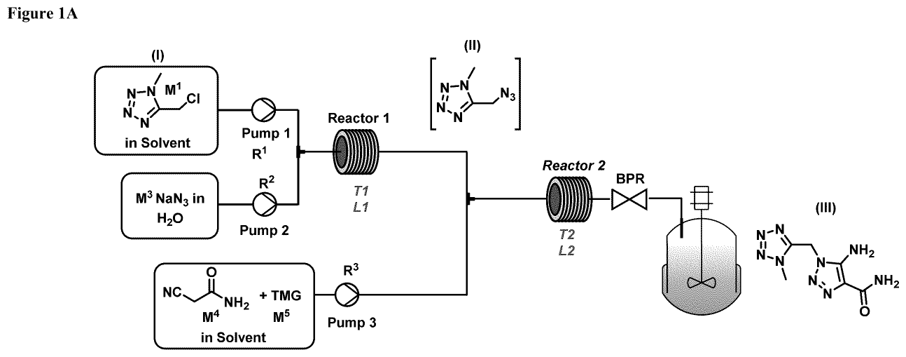

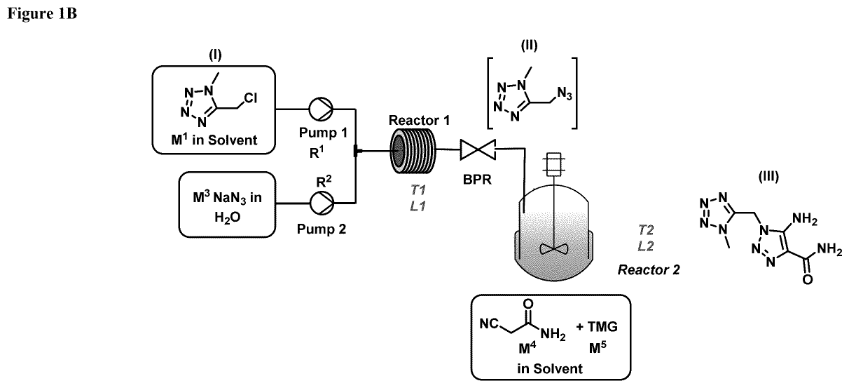

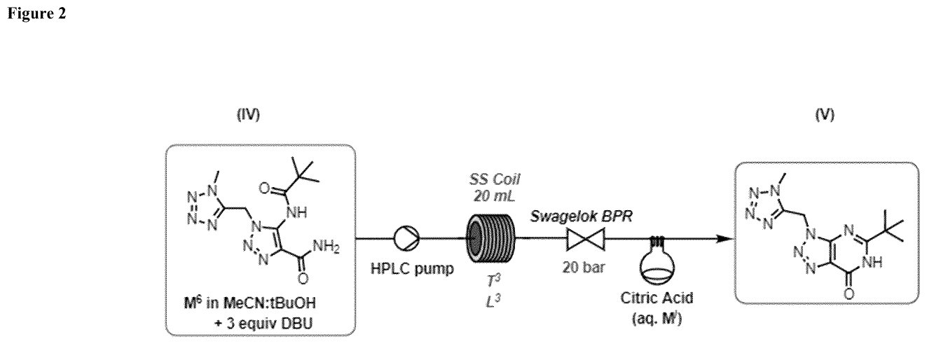

R¹, R², and R³ refer to the injection rate in mL/min. According to the present invention, R¹ can be between 0.05 mL/min and 1.5 mL/min, in particular between 0.25 mL/min and 1 mL/min, R² can be between 0.05 mL/min and 1.5 mL/min, in particular between 0.1 mL/min and 0.75 mL/min, R³ can be between 0.05 mL/min and 2 mL/min, in particular between 0.4 mL/min and 1.7 mL/min. (FIG. 1A).

L¹ and L² refer to the reaction time in minutes. According to the present invention, L¹ can be between 1 and 20 minutes, in particular between 1 and 6 minutes, more particularly 4 minutes, L² can be between 5 and 20 minutes, particularly between 8 and 15 minutes, more particularly between 10 and 15 minutes (FIG. 1A); or L² can be between 0.5 hours and 28 days particularly between 12 hours and 5 days, more particularly between 1 and 2 days (FIG. 1B).

M¹, M², M³, M⁴, and M⁵ refer to molarity expressed in M. According to the present invention, M¹ can be between 0.5 M and 2 M, in particular between 1 M and 2M, M² can be between 1 M and 3 M, in particular between 2 M and 3 M, M³ can be between 1 M and 3 M, in particular between 2 M and 3 M, M⁴ can be between 0.5 M and 2 M, in particular between 1 M and 2M, M⁵ can be between 0.5 M and 2 M, in particular between 1 M and 2M,

Compound of formula (I) is 1 equivalent, NaN3 is between 0.6 and 3 equivalents, particularly 1.05 equivalents. 2-Cyanoacetamide is between 1 and 5 equivalents, particularly 1.3 equivalents, TMG is between 0.2 and 2 equivalents, in particular 1.05 equivalents.

FIG. 2 illustrates the flow process of synthesis from compound of formula IV to compound of formula V. T³ refers to temperature in ° C. According to the present invention, T³ can be between 100° C. and 250° C., in particular between 120° C. and 230° C., more particularly between 150° C. and 200° C. L³ refers to the reaction time in minutes. According to the present invention, L³ can be between 1 and 20 minutes, in particular between 5 and 20 minutes, more particularly 15 minutes. M⁶, and M⁷ refer to molarity expressed in M. According to the present invention, M⁶ can be between 0.05 M and 2 M, in particular between 1 M and 2M, more particularly 0.5 M, M⁷ can be between 1 M and 4 M, in particular between 3 M and 4 M, more particularly 3.2 M.

In particular, between 1.0 and 5.0 equivalents of DBU with respect to compound of formula (IV) are used, in particular 3.0 equivalents are used.

Unless otherwise stated, the following terms used in the specification and claims have the meanings given below:

“ambient conditions” or “Room Temperature” refers to conditions as experienced in a standard laboratory, e.g. atmospheric pressure, under air, Ar or N₂, ambient temperature between 18° C. and 28° C.

“Compound of formula (VI)” refers to

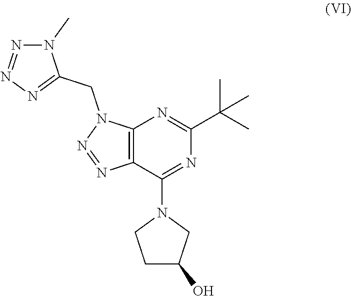 [SMILES: Cn1nnnc1Cn1nnc2c(N3CC[C@H](O)C3)nc(C(C)(C)C)nc21]

“Compound of formula (VI)” is also known as rac-(3S)-1-[5-tert-butyl-3-[(1-methyltetrazol-5-yl)methyl] triazolo[4,5-d]pyrimidin-7-yl]pyrrolidin-3-ol. Herein compound of formula (VI)'s name or reference can be interchangeably used.

The term “inorganic base” signifies alkali base, such as alkali carbonate, alkali bicarbonate, alkali borate, alkali phosphate, alkali-hydroxide. A more preferred basic aqueous solution is chosen from solution of sodium carbonate, sodium phosphate, potassium carbonate, lithium carbonate, lithium hydroxide, potassium hydroxide, sodium hydroxide, sodium hydrogen carbonate, potassium hydrogen carbonate, or lithium hydrogen carbonate, particularly NaOH, KHCO₃, NaHCO₃, K₂CO₃, Na₂CO₃, sodium phosphate, KOH, and lithium hydroxide, more particularly KHCO₃, NaHCO₃, K₂CO₃, Na₂CO₃, sodium phosphate, or a mixture thereof. The most preferred basic aqueous solution is a solution of KHCO₃, NaHCO₃, or a mixture thereof.

The term “organic base” refers to an organic Brønsted-Lowry base. Examples of organic bases are: triethylamine, 4-pyrrolidinopyridine, dimethylaminopyridine (DMAP), N-methylmorpholine, N-ethylmorpholine, pyridine, dialkylanilines, 1,1,3,3-Tetramethylguanidine (TMG), 2-picoline, 1,8-Diazabicyclo[5.4.0]undec-7-ene (DBU), sodium acetate, potassium acetate, 1,5-Diazabicyclo (4.3.0) non-5-ene (DBN), potassium acetate, imidazole, diisopropylamine, diisopropylcyclohexylamine, in particular according to the invention the organic base is 1,1,3,3-Tetramethylguanidine (TMG), pyridine, 2-picoline, 1,8-Diazabicyclo[5.4.0]undec-7-ene (DBU), sodium acetate, potassium acetate, 1,5-Diazabicyclo (4.3.0) non-5-ene (DBN), potassium acetate, imidazole, or diisopropylamine, more particularly 1,1,3,3-Tetramethylguanidine (TMG), 1,8-Diazabicyclo[5.4.0]undec-7-ene (DBU), or pyridine.

The term “Brønsted-Lowry base” refers to any chemical species that is capable of accepting a proton.

The term “Base” refers to any Brønsted-Lowry base, any organic base, or any inorganic base. Examples of bases are 1,1,3,3-Tetramethylguanidine (TMG), 2-picoline, 1,8-Diazabicyclo[5.4.0]undec-7-ene (DBU), sodium acetate, potassium acetate, 1,5-Diazabicyclo (4.3.0) non-5-ene (DBN), potassium acetate, imidazole, diisopropylamine, pyridine, KHCO₃, NaHCO₃, K₂CO₃, Na₂CO₃, or sodium phosphate.

The term “tautomer” means constitutional isomers that undergo such rapid interconversion that they cannot be independently isolated.

The term “pivaloyl source” refers to a molecule able to produce an electrophilic pivaloyl. Examples of pivaloyl sources are pivalic acid, pivaloyl chloride, or pivalic anhydride, in particular pivaloyl chloride.

The term “salt” denotes those salts formed with inorganic acids such as hydrochloric acid, hydrobromic acid, sulfuric acid, carbonic acid, formic acid, acetic acid, phosphoric acid, and organic acids selected from aliphatic, cycloaliphatic, aromatic, araliphatic, heterocyclic, carboxylic, and sulfonic classes of organic acids such as methanesulfonic acid, ethanesulfonic acid, and p-toluenesulfonic acid, in particular salt refers to salts formed with hydrochloric acid and citric acid.

The terms “hydroxyl” and “hydroxy”, alone or in combination, signify the —OH group.

The term “telescoping” means the execution of multiple reaction steps (including quenches and other workup operations) without the direct isolation of intermediates.

The term “chlorine source” means any chemical reagent that can be used to add chlorine atoms to other chemicals. In particular, the chlorine source can be POCl₃, PCl₃, calcium hypochlorite, oxalyl chloride, PCl₅, Cl₂, thionyl chloride, trichloroisocyanuric acid, more particularly the chlorine source is oxalyl chloride or POCl₃.

The azide source refers to a metal azide or organic azide. In particular, the metal azide can be an alkali metal such as potassium, sodium, lithium, rubidium or cesium. The metal can be a transition metal such as, but not limited to, iron, cobalt, nickel, copper or zinc. It is understood that certain metal azides can be formed in solution by mixing sodium azide or the like with a metal salt. In particular, a transition metal salt, such as copper sulphate. The azide of the present invention can also be an organic azide or ammonium azide. Some metal azides such as Cu(N₃)₂ and Pb(N₃)₂, are explosive and if such azides are used, precautions should be taken (e.g., that copper-containing fittings, such as brass or bronze, should not be used). Examples of azide according to the invention are selected from sodium azide, trimethylsilyl azide, tetrabutylammonium azide, or benzenesulfonyl azide or a combination thereof, in particular sodium azide.

As used herein, the terms “continuous flow process”, “continuous flow processes”, “flow process”, “continuous process” and “semi-continuous process” are used interchangeably to refer to a chemical process that utilizes flow chemistry and technology. Both single step and multiple step chemical reactions can be conducted using flow chemistry. Those skilled in the art recognize that flow chemistry involves the use of channels or tubing to conduct a chemical reaction (or series of chemical reactions) in a continuous stream rather than in separate batches using traditional vessels such as reaction flasks. Those skilled in the art are also aware of various kinds of continuous flow reactors in which flow chemistry may be conducted, such as tubular reactors (including spinning tube reactors), microreactors, spinning disk reactors, multi-cell flow reactors, CSTRs, oscillatory flow reactors, hex reactors and aspirator reactors. A continuous flow process can be scaled up or down, and therefore does not necessarily imply a particular continuous flow reactor size. In various embodiments the channels or tubing of the continuous flow reactor have a cross-sectional size (e.g., diameter for a tube having a circular cross-section) that is in the range of 1.5 mm to about 51 mm. Thus, examples of cross-sectional size (e.g., diameter) for the channels or tubes of the include the following: about 1.5 mm or greater, about 3 mm or greater, about 6 mm or greater, about 9 mm or greater, about 13 mm or greater, about 25 mm or greater, about 51 mm or less, about 25 mm or less, about 22 mm or less, about 19 mm or less, about 16 mm or less, about 13 mm or less, about 9 mm or less, or about 6 mm or less. Those skilled in the art will understand that the aforementioned descriptions of channel or tubing sizes provide a description of ranges between suitable combinations, e.g., from about 3 mm to about 6 mm. The terminology used herein with respect to continuous flow processes, flow chemistry and flow equipment is to be understood as having the ordinary meaning known to those skilled in the art. See M. B. Plutschack et al., “The Hitchhiker's Guide to Flow Chemistry” Chem. Rev. (June 2017), which is hereby incorporated by reference and particularly for the purpose of describing various continuous flow processes, flow chemistries, flow techniques and flow equipment. For any particular continuous flow process, scaling up or down can be accomplished by utilizing a continuous flow reactor having a larger or smaller tubing diameter, respectively. Scale up or down can also be achieved by increasing or decreasing the number of continuous flow reactors used to carry out the continuous flow. Reactor techniques and conditions, such as mixing, pressure, temperature, flow rate, reaction rate, reaction time and/or extent of reaction, can be controlled and/or monitored using known techniques and equipment such as vessels, CSTRs, tubing, pumps, valves, mixers, back pressure regulators (BPR), coolers, heaters, temperature sensors, temperature regulators, reaction monitors (such as in-line flow infrared (IR) monitor), photo reactors (e.g., equipped with UV source such as mercury lamp or 365 nm UV LED), membrane separators and computers. Those skilled in the art can control and monitor reactor conditions using routine experimentation informed by the detailed guidance and working examples provided herein.

“Batch reaction” or “batch” means the reactants are mixed together and allowed to react until the reactants are completely converted to product.

“Batch reactor” means a receptacle suitable to carry out a batch reaction in a stirred tank. Those skilled in the art are also aware of various kinds of batch reactors in which batch chemistry may be conducted as described in the art.

In some embodiments, a method for forming a tetrazole formula (II) is conducted in a flow reactor. Flow reactors will be known to those of ordinary skill in the art. Flow reactors may be provided in various configurations and may be equipped with a number of components to utilize methods described herein. Non-limiting components of a flow reactor include inlet(s) (e.g., for reactants, solvents, quenching agents, etc.), reaction tube and/or chamber (e.g., where the reaction occurs), outlet(s), pressure controller(s) (e.g., back pressure regulators), and temperature control device(s) (e.g., heating device(s) and/or cooling device(s)). A non-limiting example of a flow reactor is shown in FIGS. 1A and 1B and is described in more detail herein.

In another embodiment, the present invention provides a process for the preparation of compound of formula (II)

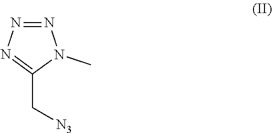

which comprises reacting compound of formula (I)

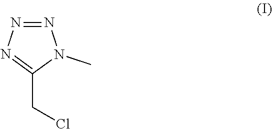

in the presence of an azide source.

In another embodiment, the present invention provides a process for the preparation of compound of formula (III)

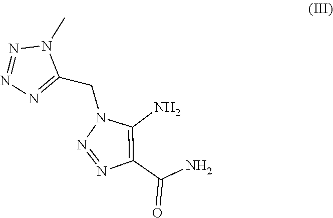 [SMILES: Cn1nnnc1Cn1nnc(C(N)=O)c1N]

which comprises reacting compound of formula (II)

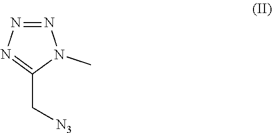

with 2-cyanoacetamide.

In another embodiment, the present invention provides a process for the preparation of compound of formula (IV)

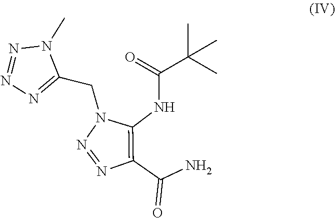 [SMILES: Cn1nnnc1Cn1nnc(C(N)=O)c1NC(=O)C(C)(C)C]

which comprises reacting compound of formula (III)

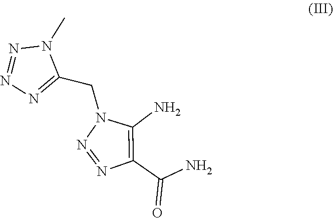 [SMILES: Cn1nnnc1Cn1nnc(C(N)=O)c1N]

with a pivaloyl source.

In another embodiment, the present invention provides a process for the preparation of compound of formula (V)

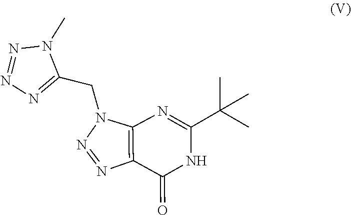 [SMILES: Cn1nnnc1Cn1nnc2c(=O)[nH]c(C(C)(C)C)nc21]

which comprises reacting compound of formula (IV)

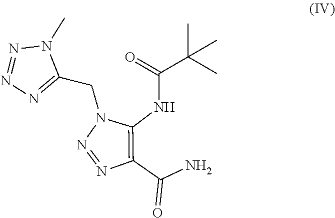 [SMILES: Cn1nnnc1Cn1nnc(C(N)=O)c1NC(=O)C(C)(C)C]

with a base.

In another embodiment, the present invention provides a process for the preparation of compound of formula (VI)

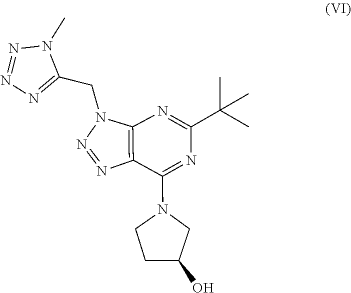 [SMILES: Cn1nnnc1Cn1nnc2c(N3CC[C@H](O)C3)nc(C(C)(C)C)nc21]

which comprises reacting compound of formula (V)

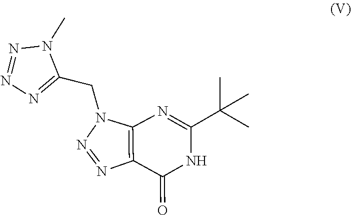 [SMILES: Cn1nnnc1Cn1nnc2c(=O)[nH]c(C(C)(C)C)nc21]

with compound of formula (VII)

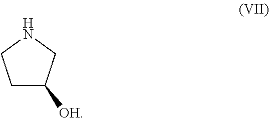

In another embodiment, the present invention provides a process for the preparation of compound of formula (VI)

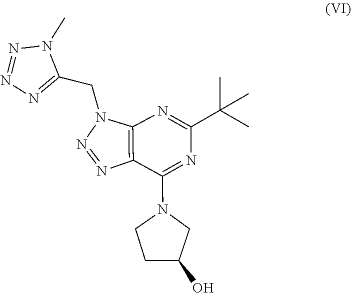 [SMILES: Cn1nnnc1Cn1nnc2c(N3CC[C@H](O)C3)nc(C(C)(C)C)nc21]

which comprises reacting compound of formula (V)

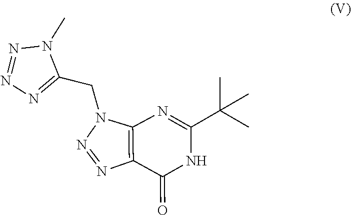 [SMILES: Cn1nnnc1Cn1nnc2c(=O)[nH]c(C(C)(C)C)nc21]

with compound of formula (VII)

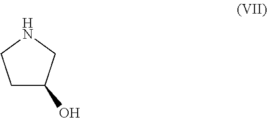

in the presence of a chlorine source, in particular oxalyl chloride or POCl₃.

In another embodiment, the present invention provides a process for the preparation of compound of formula (VI)

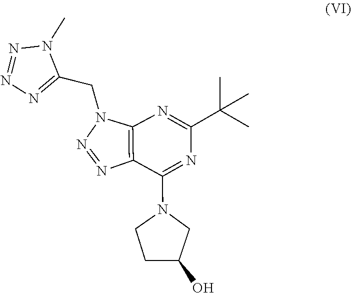 [SMILES: Cn1nnnc1Cn1nnc2c(N3CC[C@H](O)C3)nc(C(C)(C)C)nc21]

which comprises reacting compound of formula (VIII)

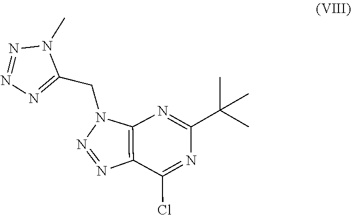 [SMILES: Cn1nnnc1Cn1nnc2c(Cl)nc(C(C)(C)C)nc21]

with compound of formula (VII)

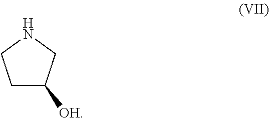

In another embodiment, the present invention provides a process for the preparation of compound of formula (VI)

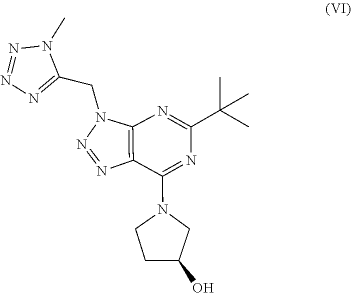 [SMILES: Cn1nnnc1Cn1nnc2c(N3CC[C@H](O)C3)nc(C(C)(C)C)nc21]

which comprises:

reacting compound of formula (V)

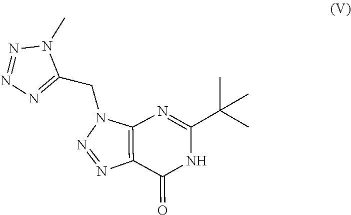 [SMILES: Cn1nnnc1Cn1nnc2c(=O)[nH]c(C(C)(C)C)nc21]

in the presence of a chlorine source, in particular oxalyl chloride or POCl₃ to form compound of formula (VIII);

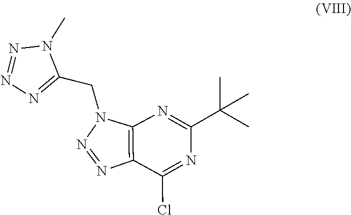 [SMILES: Cn1nnnc1Cn1nnc2c(Cl)nc(C(C)(C)C)nc21]

reacting said compound of formula (VIII) with compound of formula (VII)

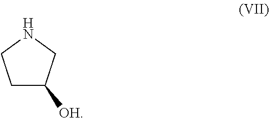

In another embodiment, the present invention provides a process for the preparation of compound of formula (VI)

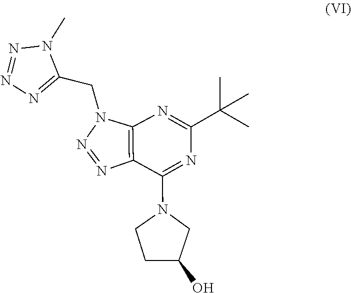 [SMILES: Cn1nnnc1Cn1nnc2c(N3CC[C@H](O)C3)nc(C(C)(C)C)nc21]

which comprises:

reacting compound of formula (IV)

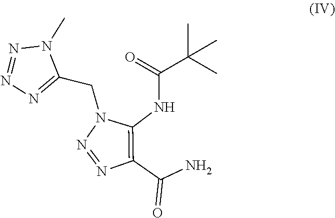 [SMILES: Cn1nnnc1Cn1nnc(C(N)=O)c1NC(=O)C(C)(C)C]

with a base to form a compound of formula (V);

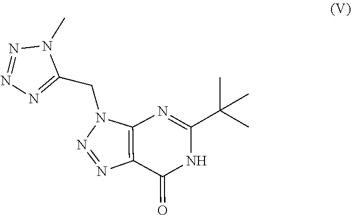 [SMILES: Cn1nnnc1Cn1nnc2c(=O)[nH]c(C(C)(C)C)nc21]

reacting said compound of formula (V) in the presence of a chlorine source, in particular oxalyl chloride or POCl₃, to form a compound of formula (VIII);

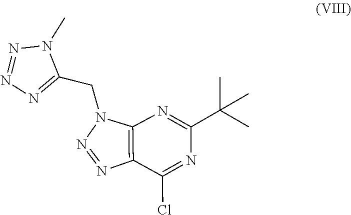 [SMILES: Cn1nnnc1Cn1nnc2c(Cl)nc(C(C)(C)C)nc21]

reacting the said compound of formula (VIII) with compound of formula (VII)

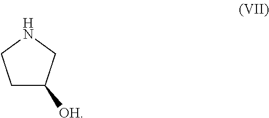

In another embodiment, the present invention provides a process for the preparation of compound of formula (VI)

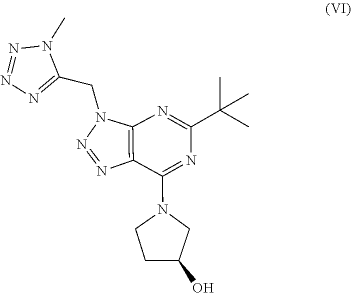 [SMILES: Cn1nnnc1Cn1nnc2c(N3CC[C@H](O)C3)nc(C(C)(C)C)nc21]

which comprises:

reacting compound of formula (III)

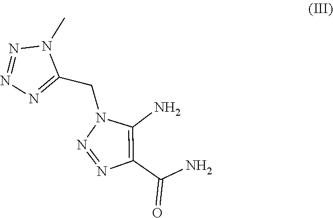 [SMILES: Cn1nnnc1Cn1nnc(C(N)=O)c1N]

with a pivaloyl source to form compound of formula (IV);

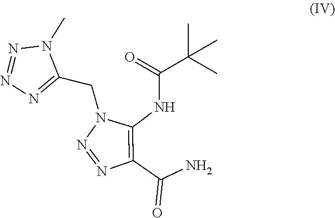 [SMILES: Cn1nnnc1Cn1nnc(C(N)=O)c1NC(=O)C(C)(C)C]

reacting said compound of formula (IV) with a base to form a compound of formula (V);

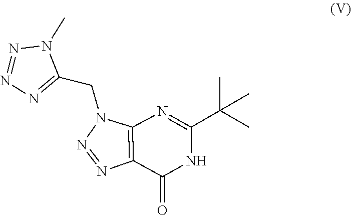 [SMILES: Cn1nnnc1Cn1nnc2c(=O)[nH]c(C(C)(C)C)nc21]

reacting said compound of formula (V) in the presence of a chlorine source, in particular oxalyl chloride or POCl₃ to form a compound of formula (VIII);

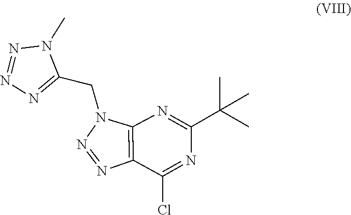 [SMILES: Cn1nnnc1Cn1nnc2c(Cl)nc(C(C)(C)C)nc21]

reacting the said compound of formula (VIII) with compound of formula (VII)

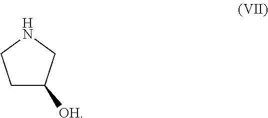

In another embodiment, the present invention provides a process for the preparation of compound of formula (VI)

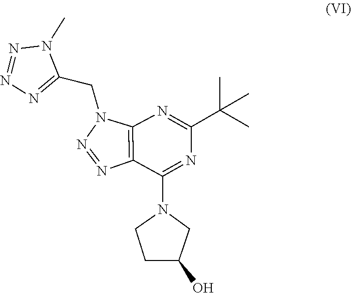 [SMILES: Cn1nnnc1Cn1nnc2c(N3CC[C@H](O)C3)nc(C(C)(C)C)nc21]

which comprises:

reacting compound of formula (II)

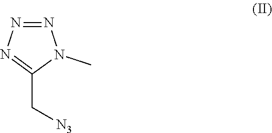

with 2-cyanoacetamide to form compound of formula (III);

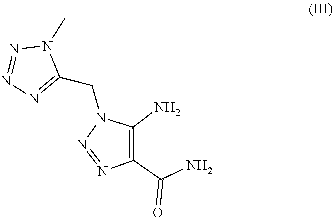 [SMILES: Cn1nnnc1Cn1nnc(C(N)=O)c1N]

reacting said compound of formula (III) with a pivaloyl source to form compound of formula (IV);

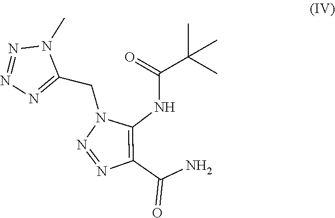 [SMILES: Cn1nnnc1Cn1nnc(C(N)=O)c1NC(=O)C(C)(C)C]

reacting said compound of formula (IV) with a base to form a compound of formula (V);

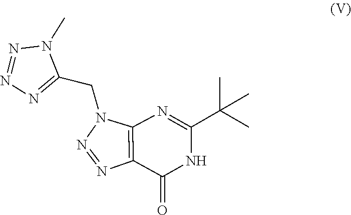 [SMILES: Cn1nnnc1Cn1nnc2c(=O)[nH]c(C(C)(C)C)nc21]

reacting said compound of formula (V) in the presence of a chlorine source, in particular oxalyl chloride or POCl₃ to form a compound of formula (VIII);

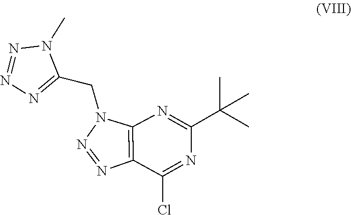 [SMILES: Cn1nnnc1Cn1nnc2c(Cl)nc(C(C)(C)C)nc21]

reacting the said compound of formula (VIII) with compound of formula (VII)

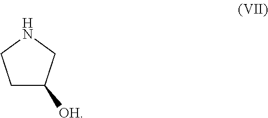

In another embodiment, the present invention provides a process for the preparation of compound of formula (VI)

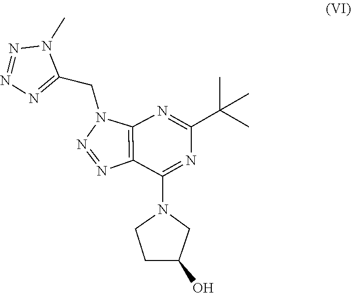 [SMILES: Cn1nnnc1Cn1nnc2c(N3CC[C@H](O)C3)nc(C(C)(C)C)nc21]

which comprises:

reacting compound of formula (I)

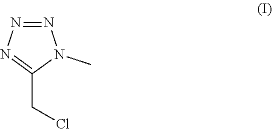

in the presence of an azide source to form compound of formula (II);

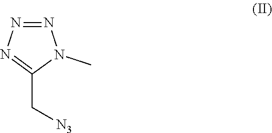

reacting said compound of formula (II) with 2-cyanoacetamide to form compound of formula (III);

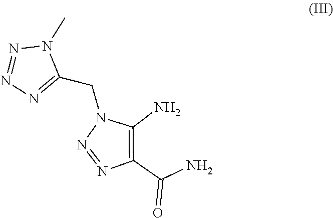 [SMILES: Cn1nnnc1Cn1nnc(C(N)=O)c1N]

reacting said compound of formula (III) with a pivaloyl source to form compound of formula (IV);

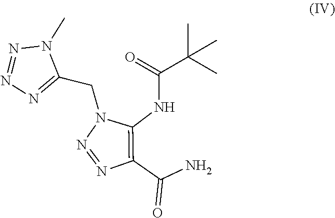 [SMILES: Cn1nnnc1Cn1nnc(C(N)=O)c1NC(=O)C(C)(C)C]

reacting said compound of formula (IV) with a base to form a compound of formula (V);

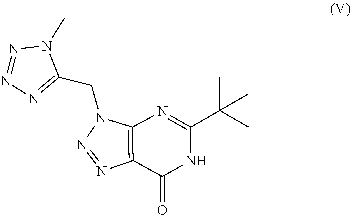 [SMILES: Cn1nnnc1Cn1nnc2c(=O)[nH]c(C(C)(C)C)nc21]

reacting said compound of formula (V) in the presence of a chlorine source, in particular oxalyl chloride or POCl₃ to form a compound of formula (VIII);

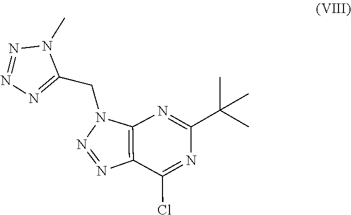 [SMILES: Cn1nnnc1Cn1nnc2c(Cl)nc(C(C)(C)C)nc21]

reacting the said compound of formula (VIII) with compound of formula (VII)

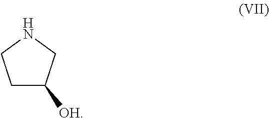

In a particular embodiment, the invention provides a multistep synthetic route as shown in scheme 1.

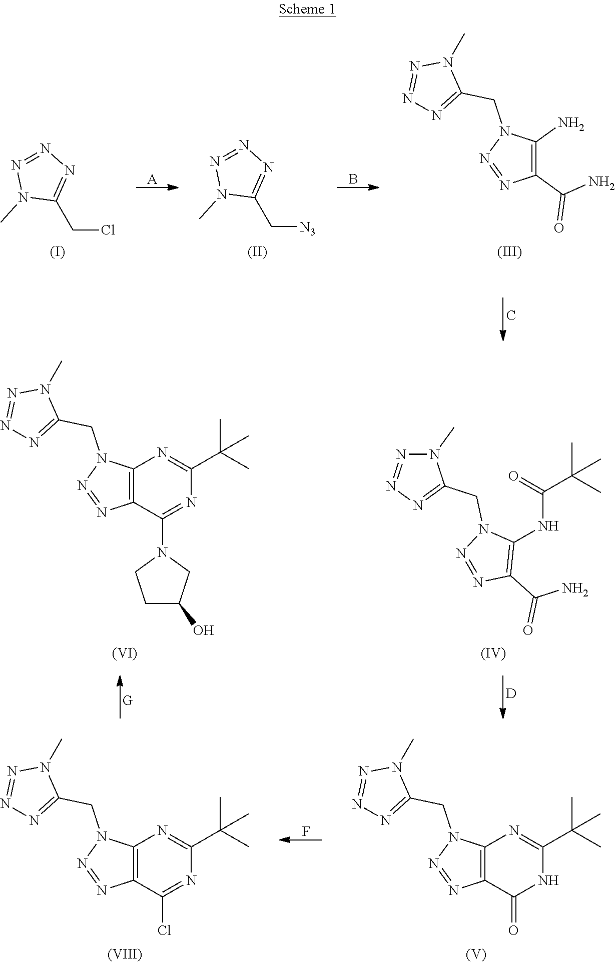

In a more particular embodiment, the invention provides a multistep synthetic route, wherein passages A-α, and C-D, as described in scheme 1, are telescoped.

In a more particular embodiment, the invention provides a multistep synthetic route, wherein passages F-G, as described in scheme 1 are telescoped.

In an even more particular embodiment, the steps A, B, and D, as described in scheme 1, are carried out in continuous flow mode, most particularly the step A is carried out in continuous flow mode.

In another embodiment, the present invention provides a process for the preparation of a compound of formula (III)

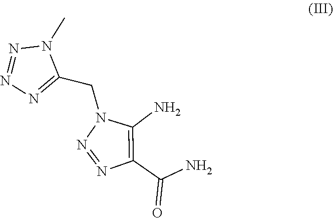 [SMILES: Cn1nnnc1Cn1nnc(C(N)=O)c1N]

which comprises:

reacting compound of formula (I)

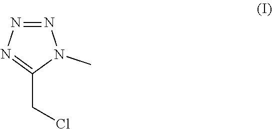

in the presence of an azide source to form compound of formula (II);

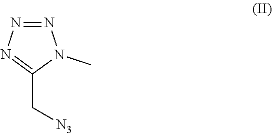

reacting said compound of formula (II) with 2-cyanoacetamide to form compound of formula (III).

In another embodiment the invention provides a process for the preparation of compound of formula (IV)

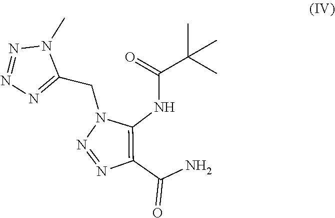 [SMILES: Cn1nnnc1Cn1nnc(C(N)=O)c1NC(=O)C(C)(C)C]

which comprises:

reacting compound of formula (I)

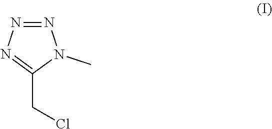

in the presence of an azide source to form compound of formula (II);

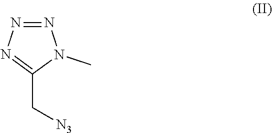

reacting said compound of formula (II) with 2-cyanoacetamide to form compound of formula (III);

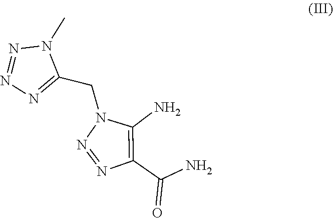 [SMILES: Cn1nnnc1Cn1nnc(C(N)=O)c1N]

reacting said compound of formula (III) with a pivaloyl source to form compound of formula (IV).

In another embodiment, the present invention provides a process for the preparation of a compound of formula (V)

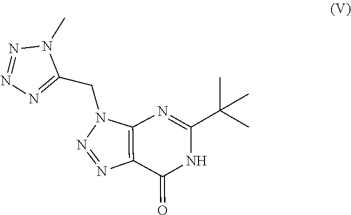 [SMILES: Cn1nnnc1Cn1nnc2c(=O)[nH]c(C(C)(C)C)nc21]

which comprises:

reacting compound of formula (I)

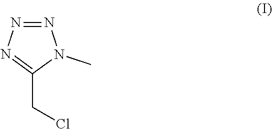

in the presence of an azide source to form compound of formula (II);

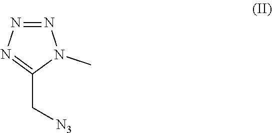

reacting said compound of formula (II) with 2-cyanoacetamide to form compound of formula (III);

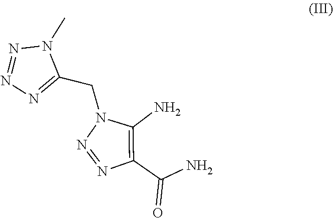 [SMILES: Cn1nnnc1Cn1nnc(C(N)=O)c1N]

reacting said compound of formula (III) with a pivaloyl source to form compound of formula (IV);

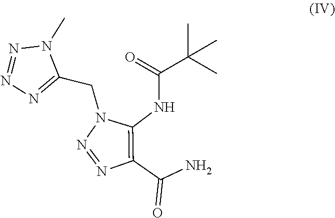 [SMILES: Cn1nnnc1Cn1nnc(C(N)=O)c1NC(=O)C(C)(C)C]

reacting said compound of formula (IV) with a base to form compound of formula (V).

In another embodiment, the present invention provides a process for the preparation of a compound of formula (VI)

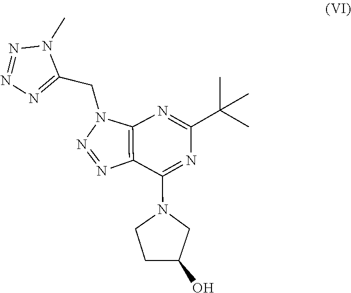 [SMILES: Cn1nnnc1Cn1nnc2c(N3CC[C@H](O)C3)nc(C(C)(C)C)nc21]

which comprises:

reacting compound of formula (I)

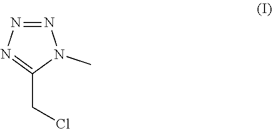

in the presence of an azide source to form compound of formula (II);

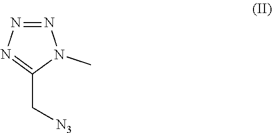

reacting said compound of formula (II) with 2-cyanoacetamide to form compound of formula (III);

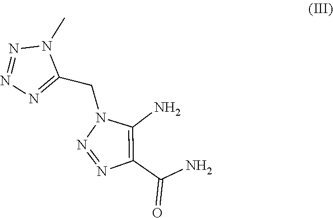 [SMILES: Cn1nnnc1Cn1nnc(C(N)=O)c1N]

reacting said compound of formula (III) with a pivaloyl source to form compound of formula (IV);

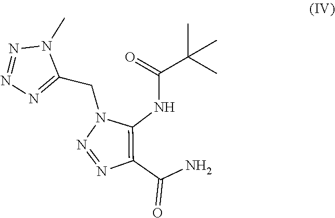 [SMILES: Cn1nnnc1Cn1nnc(C(N)=O)c1NC(=O)C(C)(C)C]

reacting said compound of formula (IV) with a base to form compound of formula (V);

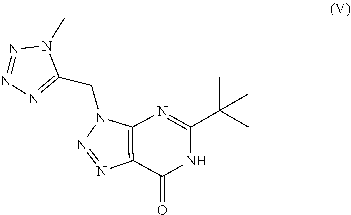 [SMILES: Cn1nnnc1Cn1nnc2c(=O)[nH]c(C(C)(C)C)nc21]

reacting said compound of formula (V) with compound of formula (VII)

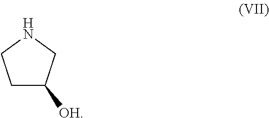

to form compound of formula (VI).

In a particular embodiment of the invention, the process or processes herein described are carried out as a continuous flow processes, in particular continuous flow processes are carried out in a flow reactor.

In other embodiment, the process or processes herein described are carried out as a semi-continuous or continuous flow processes.

In particular embodiments of the invention, the azide source is selected from, sodium azide, trimethylsilyl azide, tetrabutylammonium azide, or benzenesulfonyl azide or a combination thereof, in particular sodium azide.

In another embodiment, the present invention provides a process for the preparation of compound of formula (II) which comprises reacting compound of formula (I) in the presence of an azide source and in presence of a solvent or a mixture of two solvents selected from water, dimethyl sulfoxide (DMSO), sulfolane, acetonitrile, tetrahydrofuran (THF), 2-Methyltetrahydrofuran (MeTHF), EtOH, MeOH, N-Methyl-2-pyrrolidone (NMP), N-Ethylpentedrone (NEP), dimethylacetamide (DMA), and dimethylformamide (DMF), in particular a mixture between water and EtOH.

In another embodiment, the present invention provides a process for the preparation of compound of formula (II) which comprises reacting compound of formula (I) in the presence of an azide source wherein the reaction time is between 1 and 20 minutes, in particular between 1 and 6 minutes, more particularly 4 minutes.

In another embodiment, the present invention provides a process for the preparation of compound of formula (II) which comprises reacting compound of formula (I) in the presence of an azide source, wherein the reaction temperature is between 50° C. and 150° C., particularly between 70° C. and 120° C., more particularly between 90° C. and 120° C.

In a particular embodiment of the invention, the present invention provides a process for the preparation of compound of formula (II) which comprises reacting compound of formula (I) in the presence of an azide source, wherein said process is carried out as a continuous flow processes.

In a particular embodiment of the invention, the present invention provides a process for the preparation of compound of formula (II) which comprises reacting compound of formula (I) in the presence of an azide source, wherein the reaction time is between 30 seconds and 15 minutes, particularly between 2 and 10 minutes, more particularly between 3 and 7 minutes.

In another embodiment, the present invention provides a process for the preparation of compound of formula (III) which comprises reacting compound of formula (II) with 2-cyanoacetamide, in presence of a solvent or a mixture of two solvents selected from DMSO, sulfolane, acetonitrile, water, THF, MeTHF, EtOH, NMP, NEP, MeOH, DMA, TMG, DMF, in particular a mixture between TMG and EtOH.

In another embodiment, the present invention provides a process for the preparation of compound of formula (III) which comprises reacting compound of formula (II) with 2-cyanoacetamide, wherein said process is carried out as a continuous flow processes (FIG. 1A).

In a more particular embodiment, the present invention provides a process for the preparation of compound of formula (III) which comprises reacting compound of formula (II) with 2-cyanoacetamide, wherein said process is carried out as a batch process (FIG. 1B).

In another embodiment, the present invention provides a process for the preparation of compound of formula (III) which comprises reacting compound of formula (II) with 2-cyanoacetamide, wherein said process is performed between 0° C. and 70° C., in particular between 30° C. and 50° C., more particularly at 40° C.±5° C.

In another embodiment, the present invention provides a process for the preparation of compound of formula (III) which comprises reacting compound of formula (II) with 2-cyanoacetamide, wherein the reaction time is between 5 and 20 minutes, particularly between 8 and 15 minutes, more particularly between 10 and 15 minutes (FIG. 1A) or 0.5 hours 28 days, particularly between 12 hours and 5 days, more particularly between 1 and 2 days (FIG. 1B).

In another embodiment, the present invention provides a process for the preparation of compound of formula (III) which comprises reacting compound of formula (II) with 2-cyanoacetamide, in presence of a base selected from TMG, pyridine, 2-picoline, DBU, and NaHCO₃, in particular TMG, pyridine, and 2-picoline, more particularly TMG.

In another embodiment, the present invention provides a process for the preparation of compound of formula (IV) which comprises reacting compound of formula (III) with a pivaloyl source, in the presence of acetonitrile, pyridine, 1,3-dimethylimidazolydin-2-one (DMI), ethyl acetate, sulfolane, diethyl carbonate, methyl t-butyl ether, i-propyl acetate, n-propyl acetate, or tetrahydrofuran.

In another embodiment, the present invention provides a process for the preparation of compound of formula (IV) which comprises reacting compound of formula (III) with a pivaloyl source, in presence of acetonitrile, pyridine, ethyl acetate, sulfolane, diethyl carbonate, methyl t-butyl ether, i-propyl acetate, n-propyl acetate, or tetrahydrofuran or a mixture thereof, in particular in the presence of pyridine and acetonitrile.

In a more particular embodiment, the present invention provides a process for the preparation of compound of formula (IV) which comprises reacting compound of formula (III) with a pivaloyl source in presence of DMI.

In another embodiment, the present invention provides a process for the preparation of compound of formula (IV) which comprises reacting compound of formula (III) with a pivaloyl source, wherein the pivaloyl source is selected from pivalic acid, pivaloyl chloride, or pivalic anhydride, in particular pivaloyl chloride.

In another embodiment, the present invention provides a process for the preparation of compound of formula (IV) which comprises reacting compound of formula (III) with a pivaloyl source, wherein the reaction temperature is between 100° C. and 250° C., particularly between 130° C. and 230° C., more particularly between 150° C. and 200° C.

In another embodiment, the present invention provides a process for the preparation of compound of formula (IV) which comprises reacting compound of formula (III) with a pivaloyl source, wherein the reaction time is between 5 and 45 minutes, particularly between 8 and 25 minutes, more particularly between 10 and 20 minutes.

In another embodiment, the present invention provides a process for the preparation of compound of formula (V) which comprises reacting compound of formula (IV) with a base, wherein the base is selected from Brønsted-Lowry base, any organic base, or any inorganic base, particularly the base is selected from 1,8-Diazabicyclo[5.4.0]undec-7-ene (DBU), pyridine, sodium acetate, potassium acetate, DBN, KHCO₃, NaHCO₃, K₂CO₃, Na₂CO₃, potassium acetate, imidazole, sodium phosphate, triethylamine, diisopropylamine, more particularly wherein the base is DBU or pyridine.

In another embodiment, the present invention provides a process for the preparation of compound of formula (V) which comprises reacting compound of formula (IV) with a base, wherein said process is performed between 100° C. and 250° C., in particular between 120° C. and 230° C., more particularly between 150° C. and 200° C.

In another embodiment, the present invention provides a process for the preparation of compound of formula (VI) which comprises reacting compound of formula (V) with compound of formula (VII), in presence of DMF, DMSO, DMA, pyridine, acetonitrile, ethyl acetate, diethyl carbonate, diethyl ether, methyl t-butyl ether, isopropyl acetate, n-propyl acetate, or tetrahydrofuran or a mixture thereof, in particular in the presence of DMF and acetonitrile.

In another embodiment, the present invention provides a process as shown in scheme 2

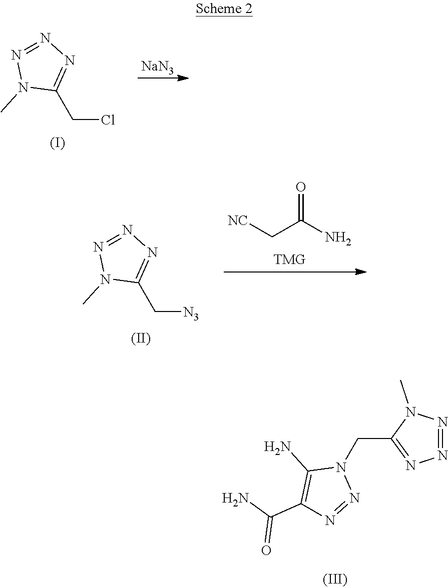

wherein compound of formula (I) is 1 equivalent, NaN₃ is between 0.6 and 3 equivalents, particularly 1.05 equivalents, 2-Cyanoacetamide is between 1 and 5 equivalents, particularly 1.3 equivalents, TMG is between 0.2 and 2 equivalents, in particular 1.05 equivalents.

In another embodiment, the present invention provides a process as shown in scheme 3

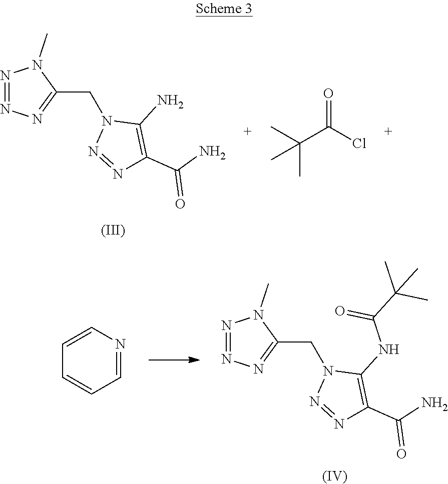

wherein compound of formula (III) is 1 equivalent, pivaloyl chloride is between 0.8 and 3 equivalents, in particular 1.25 equivalent, pyridine is between 0.2 and 3 equivalents, in particular 0.5 equivalents.

In another embodiment, the present invention provides a process as shown in scheme 6

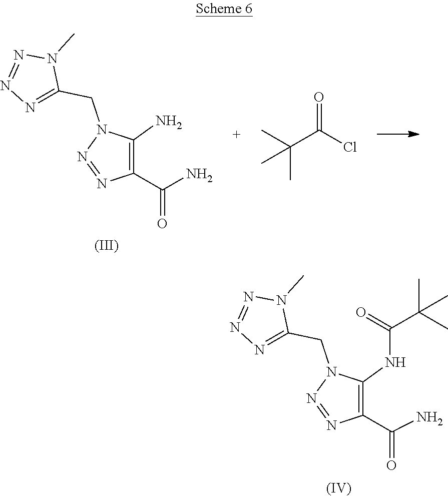

Wherein compound of formula (III) is 1 equivalent, pivaloyl chloride is between 0.8 and 3 equivalents, in particular 1.5 equivalents.

In another embodiment, the present invention provides a process as shown in scheme 4

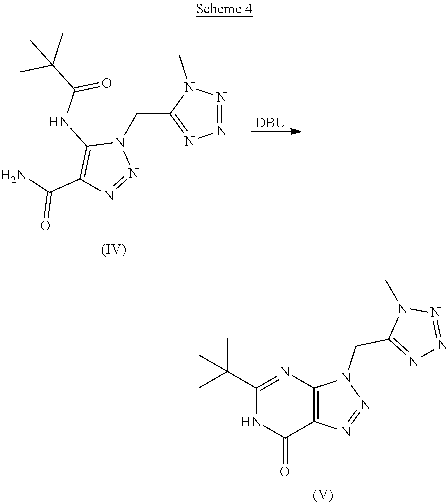

wherein compound of formula (IV) is 1 equivalent, DBU is between 1 and 5 equivalents, in particular 3 equivalents.

In a particular embodiment, the invention provides a multistep synthetic route as shown in scheme 5.

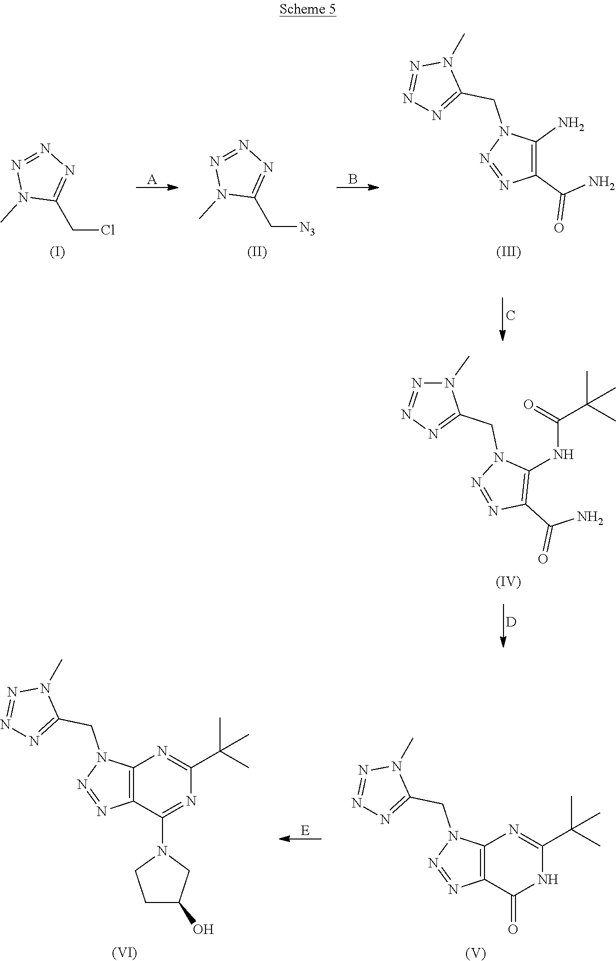

In a more particular embodiment, the invention provides a multistep synthetic route, wherein passages A-B, and C-D, as described in scheme 5, are telescoped.

In an even more particular embodiment, the steps A, B, and D, as described in scheme 5, are carried out in continuous flow mode, most particularly the step A is carried out in continuous flow mode.

The starting materials, reagents and catalysts, in particular compound of formula (I), which do not have their synthetic route explicitly disclosed herein, are generally available from commercial sources or are readily prepared using methods known to the person skilled in the art.

In another embodiment, the present invention provides a compound of formula (III) or a salt thereof

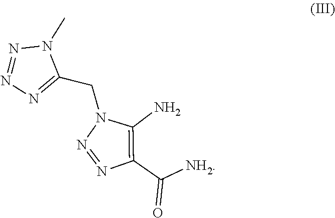 [SMILES: Cn1nnnc1Cn1nnc(C(N)=O)c1N]

In another embodiment, the present invention provides a compound of formula (IV) or a salt thereof

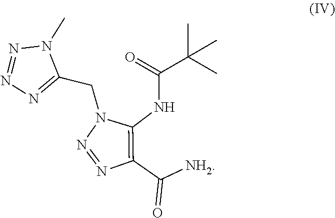 [SMILES: Cn1nnnc1Cn1nnc(C(N)=O)c1NC(=O)C(C)(C)C]

In another embodiment, the present invention provides a compound of formula (V) or a salt thereof

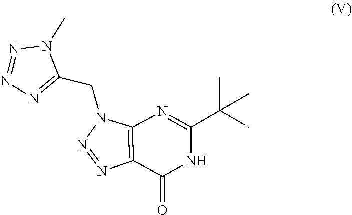

In general, the nomenclature used in this Application is based on MDL.Draw. Editor 22.1.3, a Biovia Draw computerized system for the generation of IUPAC systematic nomenclature. Chemical structures shown herein were prepared using MDL.Draw.Editor 22.1.3.1260. Any open valency appearing on a carbon, oxygen or nitrogen atom in the structures herein indicates the presence of a hydrogen atom.

The following examples are provided for illustration of the invention. They should not be considered as limiting the scope of the invention, but merely as being representative thereof. In the present application, the following abbreviations and definitions are used: br (broad); CDCl₃ (deuterated chloroform); d (doublet); DMA (Dimethylacetamide); DMAP (4-dimethylaminopyridine); TMG (1,1,3,3-Tetramethylguanidine); DBU (1,8-Diazabicyclo[5.4.0]undec-7-ene); DBN (1,5-Diazabicyclo (4.3.0) non-5-ene); DMSO (dimethyl sulfoxide); THF (tetrahydrofuran); MeTHF (2-Methyltetrahydrofuran); EtOH (ethanol); NEP (N-Ethylpentedrone); DMF (Dimethylformamide); eq. (equivalent); g (gram); GC (gas chromatography); h (hour); H₂O (water); HPLC (High-Performance Liquid Chromatography); iPrOH (isopropanol); KOH (Potassium Hydroxide); LCMS (Liquid chromatography-mass spectrometry); M (Molar); m (multiplet); MeOH (methanol); MS (Mass Spectroscopy); mL (milliliter); NaOH (Sodium hydroxide); NMP (N-Methyl-2-Pyrrolidone); NMR (nuclear magnetic resonance); s (singlet); see (second); t (triplet); THF (tetrahydrofuran).

### Example 1: Synthesis of 5-amino-1-[(1-methyltetrazol-5-yl)methyl] triazole-4-carboxamide

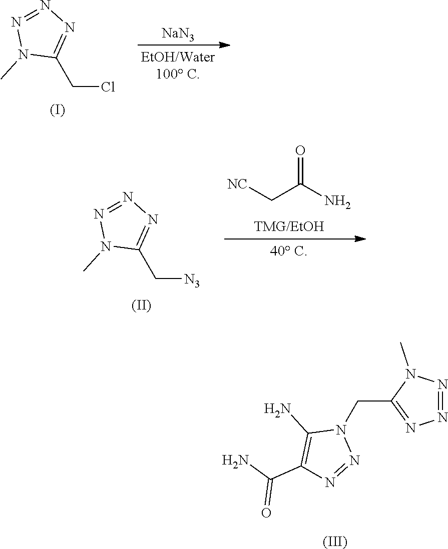

In FIG. 1A is shown the flow setup for the formation of the (III) with the continuous flow approach and in FIG. 1B formation of (II) in the continuous flow and formation of (III) in the batch reactor. Further lab procedure reports scenario depicted in FIG. 1B.

### Feed Preparation:

1.30 M of 5-(chloromethyl)-1-methyl-1H-tetrazole (Cl-tetrazole): In a 250 mL volumetric flask, Cl-tetrazole (43.08 g, 325.01 mmol) was dissolved and filled up to the 250 mL mark with ethanol

2.00 M of NaN₃: In a 100 mL volumetric flask sodium azide (13.13 g, 622.77 mmol) was dissolved and filled up to the 100 mL mark with water. Additional 20 μL of NaOH were added to ensure pH>8.

### Reaction:

In a 100 mL batch Reactor 2 add 2-cyanoacetamide (5.642 g, 1.30 equiv) followed by the TMG (6.2428 g, 1.05 equiv) and ethanol (45 mL) to form a light suspension. Warm reaction mixture to JT 40° C.

Start addition of Cl-tetrazole feed solution (0.35 mL/min, 1.0 equiv)) and NaN₃ (feed (0.25 mL/min, 1.1 equiv) in the reactor 1. Reactor 1 consists of a T-piece (PEEK, 0.8 mm i.d.). The output of the mixer was connected to a residence time unit (5 min, PFA tubing, 0.8 mm i.d.). Reactor 1 JT is 100° C. and residence time of 5 minutes. The output of a reactor 1 is connected to a back pressure regulator (Zaiput BPR-10) which was set to 3-4 bars. The output of the reactor 1 is directed to the waste for 30 minutes and then to the reactor 2 over 100 minutes period. IT in the reactor 2 is 40° C. After the completion of addition reactor 2 is stirred further 0-5 h at 40° C. In Process Control (IPC) is measured to confirm full conversion.

### Work-Up and Isolation:

To the reaction mixture in reactor 2 is added water (30 mL). The formed suspension is cooled to 0° C. and stirred about 2 hours at this temperature. The suspension is filtered over 3a glass filter and washed with water (30 g) to yield a light pink product (III) (9.28 g, 81%).

### Example 2: Synthesis of 5-tert-butyl-3-[(1-methyltetrazol-5-yl)methyl]-6H-triazolo[4,5-d]pyrimidin-7-one

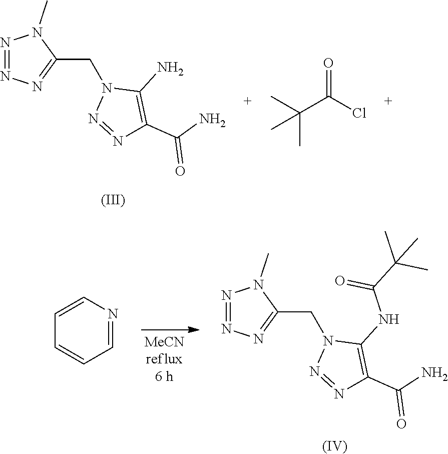

A 500 mL round bottom flask equipped with a magnetic stirring bar was charged with 5-amino-1-(1-methyl-1H-tetrazol-5-yl)methyl-1H-1,2,3-triazole-4-carboxamide (39.06 g, 175 mmol) and acetonitrile (150 mL-suspension formed). Pyridine (6.92 g, 0.5 equiv., 87.5 mmol) was added to the stirred suspension. Pivaloyl chloride (27.43 g, 1.3 equiv., 227.5 mmol) was added over 15 minutes using a dropping funnel. The addition funnel was washed with acetonitrile (25 mL). The round bottom flask was equipped with a reflux condenser and heated to reflux using an oil bath (bath temperature 95° C.). The reaction mixture cleared up to form a slightly yellow coloured homogeneous solution after around 2 h at reflux. After 6 h the reaction was cooled to room temperature using a water bath.

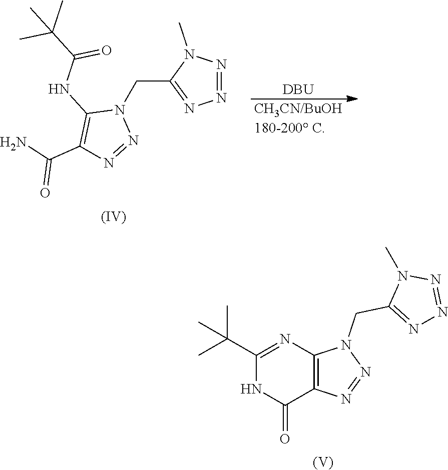

The single feed solution was pumped at 1.33 mL/min using a Knauer HPLC pump (10 mL pump head) (FIG. 2). As residence time unit, a 20 mL stainless steel coil mounted on a Uniqsis coil heater, which was heated to 190° C., was used (15 min residence time). After passing through the residence time unit, a Swagelok BPR set to 20 bar was used to enable super-heating of the reaction mixture.

### Collection of Fractions:

One non-steady state fraction was collected from 10-25 minutes after switching on the pump. Two steady state fractions (corresponding to 80 mmol each) were collected directly into stirred 250 mL round bottom flasks (equipped with stirring bars) which were charged with aqueous citric acid (3.2 M, 60 mL) quench solution each.
Fraction 1:25-145 min after start.
Fraction 2:145-265 min after start.

During the collection into the citric acid solutions, precipitation of white crystalline product started occurring around 5 minutes after collection was started.

Afterwards another non-steady state fraction was collected (265-280 min). After 260 minutes the feed was switched to acetonitrile as carrier solvent.

### Workup:

Solvents were evaporated under vacuum (40° C. water bath temperature, pressure=70 mbar). 20 mL of water was added to avoid sticking of the crystalline solid to the flask. The suspension was cooled to 4° C. overnight. The crystals were filtered and washed with 2×30 mL water, 2×30 mL i-PrOH and 3×40 mL petroleum ether. The collected white crystalline solid was dried overnight at 50° C. under vacuum. The combined steady state fractions resulted in 5-(tert-butyl)-3-((1-methyl-1H-tetrazol-5-yl)methyl)-3,6-dihydro-7H-[1,2,3]triazolo[4,5-d]pyrimidin-7-one (3 37.88 g, 82.1% yield). Roughly 0.74 g (1.6%) of product remained in the mother liquors (by HPLC analysis).

### Example 3: Synthesis of (3S)-1-[5-tert-butyl-3-[(1-methyltetrazol-5-yl)methyl]triazolo[4,5-d] pyrimidin-7-yl]pyrrolidin-3-ol

5-tert-butyl-3-[(1-methyltetrazol-5-yl)methyl]-6H-triazolo[4,5-d]pyrimidin-7-one (1.0 eq, 10.0 g) is mixed with CH₃CN (90 mL) and DMF (6.7 mL) at 20° C. The mixture is warmed to 38° C. and oxalylchloride (9.7 g, 2.2 eq) is added during 15 min. The mixture is diluted with CH₃CN (7 mL). The mixture is stirred at 38° C. for at least 4 h before cooling to 20° C. The reaction mixture is added onto a mixture of n-PrOAc (50 mL) and 8% KH₂PO₄-solution (60 mL). The phases are stirred before settling for phase separation. The organic phase is washed with 8% KH₂PO₄ (60 mL). The phases are stirred before settling for phase separation. The organic phase is washed with 5% NaHCO₃ (60 mL). The phases are stirred before settling for phase separation. The organic solution is distilled at reduced pressure and CH₃CN is constantly exchanged to n-PrOAc. To the resulting solution, DIPEA (20.0 mL, 1.3 eq) and n-PrOAc (20 ml) are added, at 30° C., followed by a solution of (3S)-pyrrolidin-3-ol (3.22 g, 1.07 eq) in water (20 mL). The mixture is stirred at 30° C. until complete consumption of starting material. The phases are separated and the organic phase is washed with water (2×30 ml). After phase separation, the organic layer is distilled under reduced pressure to a volume of 45 mL and n-heptane (16.7 g) is added. The mixture is warmed to 65° C. until all solids are dissolved before cooling to 0-5° C. The formed suspension is filtered and the filter cake is washed with n-PrOAc/n-heptane (3:2). The product is dried at 60° C. under reduced pressure to obtain the title compound as white powder (10.2 g, 82% yield.)

### Example 4: Synthesis of Compound of Formula (IV)

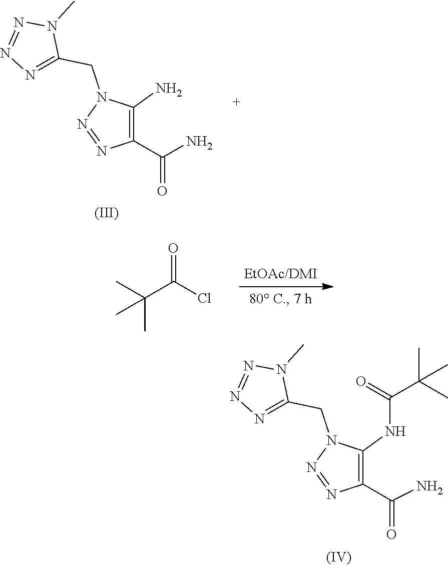

Compound of formula (III) (70 g, 313.6 mmol, 1.00 eq) is suspended in ethyl acetate (299 g) and 1,3-dimethylimidazolydin-2-one (DMI) (87.1 g) and Pivaloyl chloride (56.7 g, 470.4 mmol, 1.5 eq) is added. The mixture is heated to TR=80° C. and stirred for at least 7 h. The mixture is cooled to TR=20° C. and quenched with methanol (10.1 g, 313.6 mmol, 1.00 eq). The mixture is transferred to a suspension of sodium citrate (40.5 g, 137.61 mmol) in ethyl acetate (126 g) and treated with 25% aqueous ammonia (26.4 g, 188.2 mmol). The solvent is distilled under reduced pressure. The residue is heated to TR=65° C. and water (210 g) is slowly charged. The mixture is cooled to TR=55° C. and seeded. An aqeuopus solution of sodium citrate is slowly added. The suspension is cooled to TR=3° C. The solids are isolated by filtration and washed with cold water. The wet product is dried under reduced pressure to obtain about 87% yield.

### Example 5 Synthesis of Compound of Formula (V)

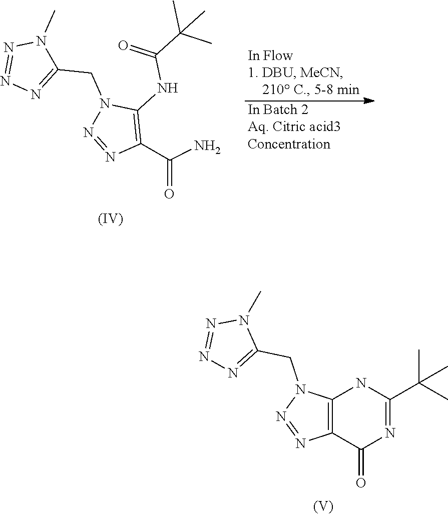

Compound of formula (IV) (60.2 g, 183.53 mmol, 1.00 eq) is dissolved in acetonitrile (141.48 ml) and DBU (47.5 g, 312 mmol, 1.70 eq) at ambient temperature. The reaction is carried out in either a batch microwave vial (Lab) or flow-through microwave (pilot/plant) at IT=210° C. for 5-8 min and ca. 20-25 bar pressure. After cooling to ambient temperature, aqueous citric acid (422.1 mmol, 2.30 eq) is added and compound of formula (V) crystallizes out of solution. Acetonitrile (103 g) is distilled off. The suspension is cooled to IT=0° C. The crystals are filtered off, washed with water (150 g) and 2-PrOH (35.1 g) and dried under vacuum (10 mbar, 45° C.) until weight constancy to give compound of formula (V) as a white solid in 89% yield.

### Aspects of the Present Invention

Aspect 1. A process for the preparation of compound of formula (II)

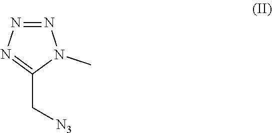

which comprises reacting compound of formula (I)

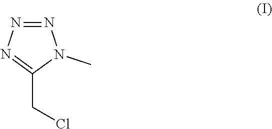

in the presence of an azide source.

Aspect 2. A process for the preparation of compound of formula (III)

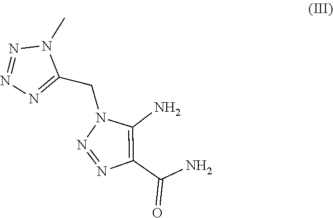 [SMILES: Cn1nnnc1Cn1nnc(C(N)=O)c1N]

which comprises reacting compound of formula (II)

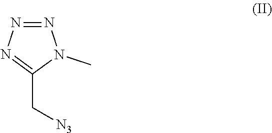

with 2-cyanoacetamide.

Aspect 3. A process for the preparation of compound of formula (IV)

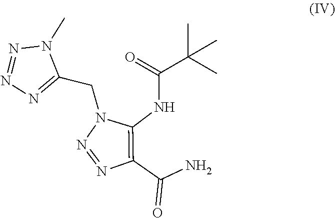 [SMILES: Cn1nnnc1Cn1nnc(C(N)=O)c1NC(=O)C(C)(C)C]

which comprises reacting compound of formula (III)

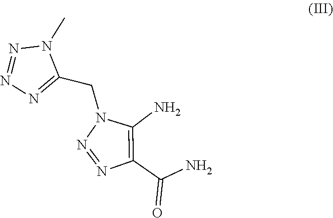 [SMILES: Cn1nnnc1Cn1nnc(C(N)=O)c1N]

with a pivaloyl source.

Aspect 4. A process for the preparation of compound of formula (V)

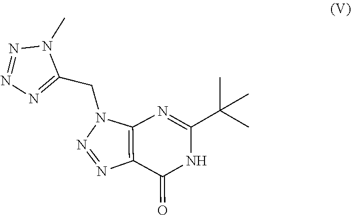 [SMILES: Cn1nnnc1Cn1nnc2c(=O)[nH]c(C(C)(C)C)nc21]

which comprises reacting compound of formula (IV)

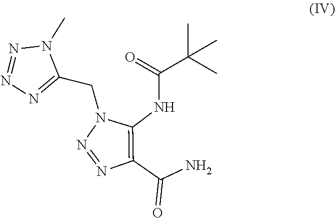 [SMILES: Cn1nnnc1Cn1nnc(C(N)=O)c1NC(=O)C(C)(C)C]

with a base.

Aspect 5. A process for the preparation of compound of formula (VI)

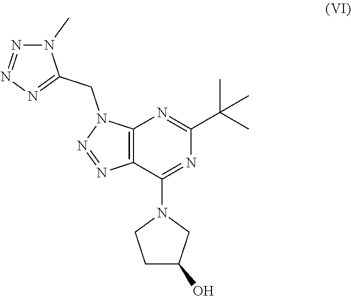 [SMILES: Cn1nnnc1Cn1nnc2c(N3CC[C@H](O)C3)nc(C(C)(C)C)nc21]

which comprises reacting compound of formula (V)

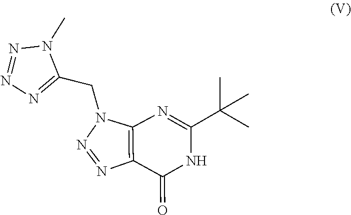 [SMILES: Cn1nnnc1Cn1nnc2c(=O)[nH]c(C(C)(C)C)nc21]

with compound of formula (VII)

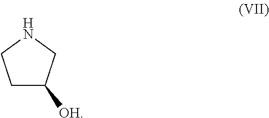

Aspect 6. The process according to aspect 1 which further comprises the preparation of a compound of formula (III)

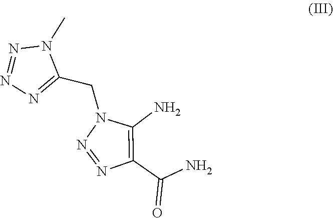 [SMILES: Cn1nnnc1Cn1nnc(C(N)=O)c1N]

which comprises reacting compound of formula (II)

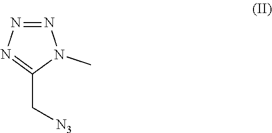

with 2-cyanoacetamide.

Aspect 7. The process according to aspects 2 or 6 which further comprises the preparation of a compound of formula (V)

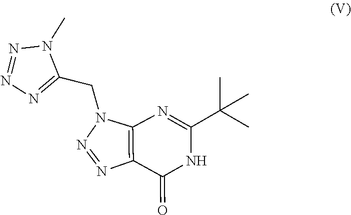 [SMILES: Cn1nnnc1Cn1nnc2c(=O)[nH]c(C(C)(C)C)nc21]

which comprises reacting compound of formula (IV)

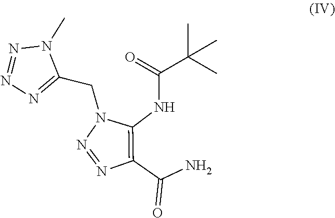 [SMILES: Cn1nnnc1Cn1nnc(C(N)=O)c1NC(=O)C(C)(C)C]

with a base.

Aspect 8. The process according to aspect 3 or 7 which further comprises the preparation of a compound of formula (VI)

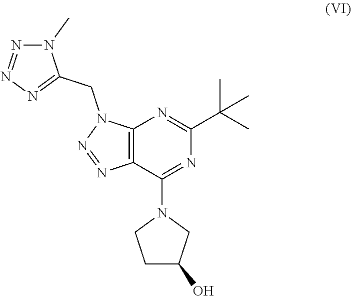 [SMILES: Cn1nnnc1Cn1nnc2c(N3CC[C@H](O)C3)nc(C(C)(C)C)nc21]

which comprises reacting compound of formula (V)

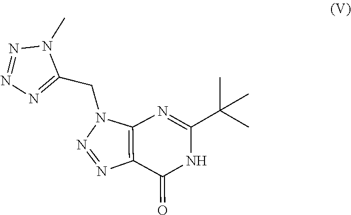 [SMILES: Cn1nnnc1Cn1nnc2c(=O)[nH]c(C(C)(C)C)nc21]

with compound of formula (VII)

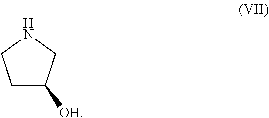

Aspect 9. The process according to aspect 6 which further comprises the preparation of compound of formula (II)

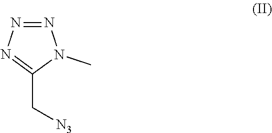

which comprises reacting compound of formula I

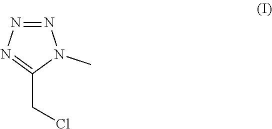

in the presence of an azide source.

Aspect 10. The process according to aspect 6 which further comprises the preparation of compound of formula III

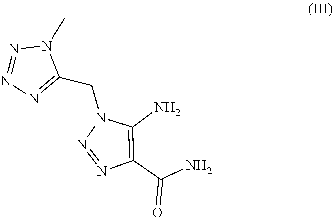 [SMILES: Cn1nnnc1Cn1nnc(C(N)=O)c1N]

which comprises reacting of compound of formula II

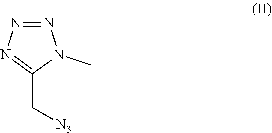

with 2-cyanoacetamide.

Aspect 11. The process according to aspect 5 which further comprises the preparation of compound of formula IV

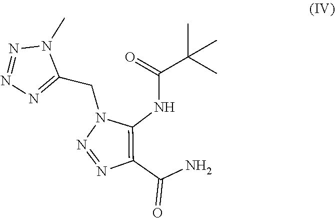 [SMILES: Cn1nnnc1Cn1nnc(C(N)=O)c1NC(=O)C(C)(C)C]

which comprises reacting of compound of formula III

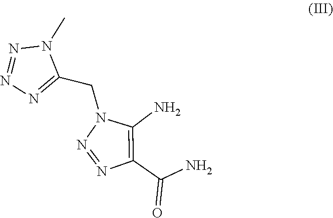 [SMILES: Cn1nnnc1Cn1nnc(C(N)=O)c1N]

with a pivaloyl source.

Aspect 12. The process according to aspect 5 which further comprises the preparation of compound of formula V

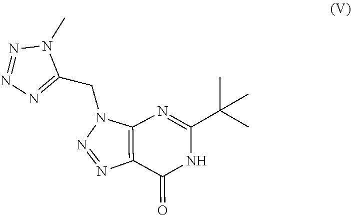 [SMILES: Cn1nnnc1Cn1nnc2c(=O)[nH]c(C(C)(C)C)nc21]

which comprises reacting of compound of formula IV

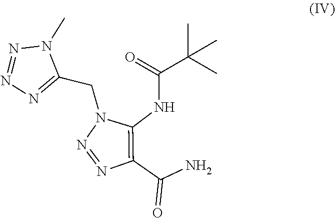 [SMILES: Cn1nnnc1Cn1nnc(C(N)=O)c1NC(=O)C(C)(C)C]

with a base.

Aspect 13. The process according to any one of the aspects 1, 2, 4, 6, 7, 9, 10, or 12 wherein the said process is carried out in flow, in particular wherein the process is carried out in a flow reactor.

Aspect 14. The process according to any one of the aspects 1, 2, 4, 6, 7, 9, 10, or 12 wherein the said process is carried out in batch, in particular wherein the process is carried out in a batch reactor.

Aspect 15. The process according to any one of the aspects 1 or 9 wherein the azide source is selected from, sodium azide, trimethylsilyl azide, tetrabutylammonium azide, or benzenesulfonyl azide or a combination thereof, in particular sodium azide.

Aspect 16. The process according to any one of the aspects 1, 9, or 15 in presence of a solvent or a mixture of two solvents selected from water, DMSO, sulfolane, acetonitrile, THF, MeTHF, EtOH, MeOH, NMP, NEP, DMA, and DMF, in particular a mixture between water and EtOH.

Aspect 17. The process according to any one of the aspects 1, 9, 15, or 16 wherein the reaction time is between 1 and 20 minutes, in particular between 1 and 6 minutes, more particularly 4 minutes.

Aspect 18. The process according to any of the aspects 1, 9, 15-17, wherein the reaction temperature is between 50° C. and 150° C., particularly between 70° C. and 120° C., more particularly between 90° C. and 120° C.

Aspect 19. The process according to any one of the aspects 2, 6, or 10 in presence of a solvent or a mixture of two solvents selected from DMSO, sulfolane, acetonitrile, water, THF, MeTHF, EtOH, NMP, NEP, MeOH, DMA, DMF, in particular EtOH.

Aspect 20. The process according to any one of the aspects 2, 6, 10, or 19 wherein said process is performed between 0° C. and 70° C., in particular between 30° C. and 50° C., more particularly at 40° C.±5° C.

Aspect 21. The process according to any one of the aspects 2, 6, 10, 19, or 20 wherein the reaction time is between 5 and 20 minutes, particularly between 8 and 15 minutes, more particularly between 10 and 15 minutes.

Aspect 22. The process according to any one of the aspects 2, 6, 10, 19, or 20 wherein the reaction time is between 0.5 hours and 28 days, particularly between 12 hours and 5 days, more particularly between 1 and 2 days in a batch process.

Aspect 23. The process according to aspect 2, 6, 10, 19-21 in presence of a base selected from TMG, pyridine, 2-picoline, DBU, and NaHCO₃, in particular TMG, pyridine, and 2-picoline, more particularly TMG.

Aspect 24. The process according to aspects 3 or 11 to 23 in the presence of acetonitrile, pyridine, DMI, ethyl acetate, sulfolane, diethyl carbonate, methyl t-butyl ether, isopropyl acetate, n-propyl acetate, or tetrahydrofuran.

Aspect 25. The process according to aspects 3, 11 to 24 in presence of acetonitrile, pyridine, ethyl acetate, sulfolane, diethyl carbonate, methyl t-butyl ether, isopropyl acetate, n-propyl acetate, or tetrahydrofuran or a mixture thereof, in particular in the presence of pyridine and acetonitrile.

Aspect 26. The process according to claims 3, 11, to 24 in the presence of DMI.

Aspect 27. The process according to any one of the aspects 3, 11 to 26, wherein the pivaloyl source is selected from pivalic acid, pivaloyl chloride, or pivalic anhydride, in particular pivaloyl chloride.

Aspect 28. The process according to any one of the aspects 3, 11 to -27, wherein the reaction temperature for steps mentioned in aspects 3, 11 or 24 is between 100° C. and 250° C., particularly between 130° C. and 230° C., more particularly between 150° C. and 200° C. Aspect 29. The process according to any one of the aspects 3, 11, 24-28, wherein the reaction time for steps mentioned in aspects 3, 11 or 24 is between 5 and 45 minutes, particularly between 8 and 25 minutes, more particularly between 10 and 20 minutes.

Aspect 30. The process according to any one of the aspects 4, 7 to 29 wherein the base for steps mentioned in aspects 4, 7 or 12 is selected from DBU, pyridine, sodium acetate, potassium acetate, DBN, KHCO₃, NaHCO₃, K₂CO₃, Na₂CO₃, potassium acetate, imidazole, sodium phosphate, triethylamine, diisopropylamine, in particular DBU or pyridine.

Aspect 31. The process according to any one of the aspects 4, 7 to 29 wherein said process is performed between 100° C. and 250° C. for steps mentioned in aspects 4, 7 or 12, in particular between 120° C. and 230° C., more particularly between 180° C. and 200° C.

Aspect 32. The process according to aspects 5 or 8, in presence of a chlorine source, in particular oxalyl chloride or POCl₃.

Aspect 33. The process according to aspects 5 or 8 in presence of a solvent or a mixture of two solvents selected from DMF, DMSO, DMA, pyridine, acetonitrile, ethyl acetate, diethyl carbonate, diethyl ether, methyl t-butyl ether, isopropyl acetate, n-propyl acetate, or tetrahydrofuran, in particular a mixture between DMF and acetonitrile.

Aspect 34. A compound of formula (III) or a salt thereof

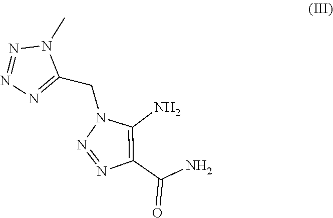 [SMILES: Cn1nnnc1Cn1nnc(C(N)=O)c1N]

Aspect 35. A compound of formula (IV) or a salt thereof

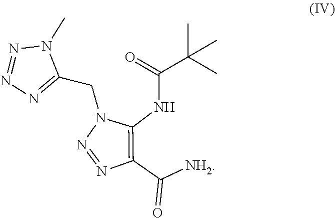 [SMILES: Cn1nnnc1Cn1nnc(C(N)=O)c1NC(=O)C(C)(C)C]

Aspect 36. A compound of formula (V) or a salt thereof

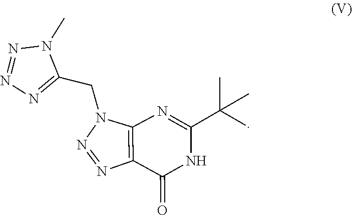

Aspect 37. A compound of formula (III)

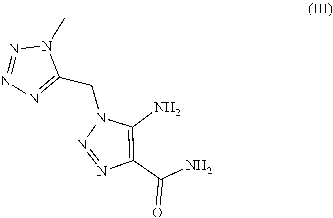 [SMILES: Cn1nnnc1Cn1nnc(C(N)=O)c1N]

Aspect 38. A compound of formula (IV)

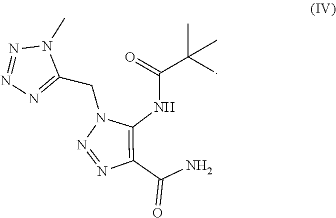

Aspect 39. A compound of formula (V)

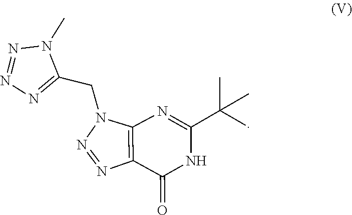

1.-2. (canceled)

3. A process for the preparation of compound of formula (III) 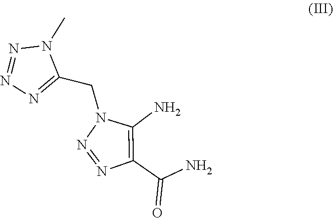 [SMILES: Cn1nnnc1Cn1nnc(C(N)=O)c1N]
which comprises reacting compound of formula (II) 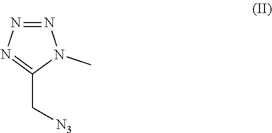
with 2-cyanoacetamide.

4. A process for the preparation of compound of formula (IV) 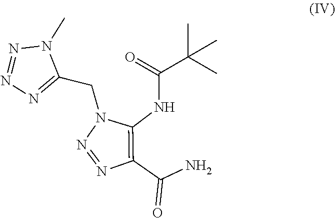 [SMILES: Cn1nnnc1Cn1nnc(C(N)=O)c1NC(=O)C(C)(C)C]
which comprises reacting compound of formula (III) 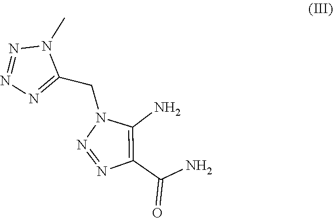 [SMILES: Cn1nnnc1Cn1nnc(C(N)=O)c1N]
with a pivaloyl source.

5. A process for the preparation of compound of formula (V) 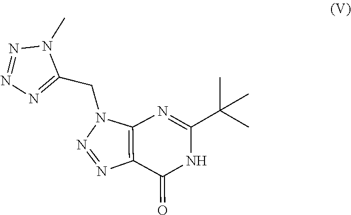 [SMILES: Cn1nnnc1Cn1nnc2c(=O)[nH]c(C(C)(C)C)nc21]
which comprises reacting compound of formula (IV) 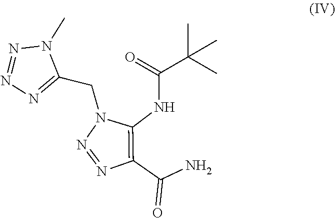 [SMILES: Cn1nnnc1Cn1nnc(C(N)=O)c1NC(=O)C(C)(C)C]
with a base.

6. The process according to claim 5
which further comprises reacting the compound of formula (V)
with a compound of formula (VII) 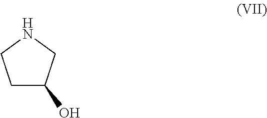
to form a compound of formula (VI)

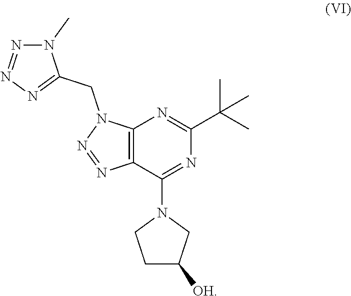 [SMILES: Cn1nnnc1Cn1nnc2c(N3CC[C@H](O)C3)nc(C(C)(C)C)nc21]

7. The process according to claim 4 which further comprises
reacting a compound of formula (II) 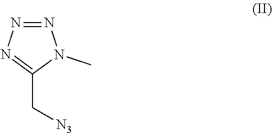
with 2-cyanoacetamide to form the compound of formula (III).

8. The process according to claim 4 which further comprises
reacting the compound of formula (IV)
with a base to form a compound of formula (V)

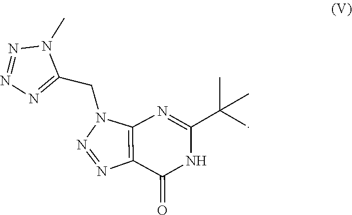

9. The process according to claim 8 which further comprises
reacting the compound of formula (V)
with a compound of formula (VII) 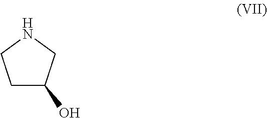
to form a compound of formula (VI)

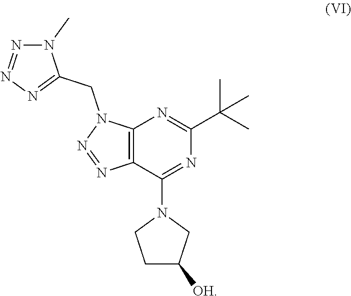 [SMILES: Cn1nnnc1Cn1nnc2c(N3CC[C@H](O)C3)nc(C(C)(C)C)nc21]

10. The process according to claim 3 which further comprises
reacting a compound of formula (I) 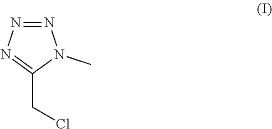
in the presence of an azide source to form the compound of formula (II).

11. The process according to claim 4 which further comprises
reacting of a compound of formula (II) 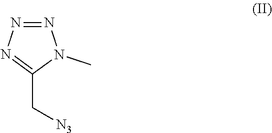
with 2-cyanoacetamide to form the compound of formula (III).

12. The process according to claim 3 which further comprises
reacting the compound of formula (III)
with a pivaloyl source to form a compound of formula (IV)

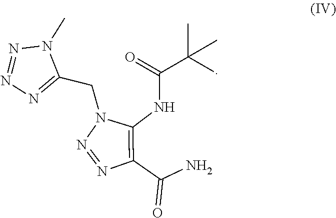

13. The process according to claim 12 which further comprises
reacting the compound of formula (IV)
with a base to form a compound of formula (V)

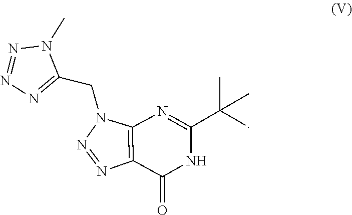

14. The process according to claim 3 wherein the process is carried out in flow.

15. The process according to claim 3 wherein the process is carried out in batch.

16. The process according to claim 10 wherein the azide source is sodium azide, trimethylsilyl azide, tetrabutylammonium azide, or benzenesulfonyl azide or a combination thereof.

17.-23. (canceled)

24. The process according to claim 3, wherein the compound of formula (II) is reacted with 2-cyanoacetamide in presence of a base selected from TMG, pyridine, 2-picoline, DBU, and NaHCO₃.

25.-30. (canceled)

31. The process according to claim 5, wherein the base is selected from DBU, pyridine, sodium acetate, potassium acetate, DBN, KHCO₃, NaHCO₃, K₂CO₃, Na₂CO₃, potassium acetate, imidazole, sodium phosphate, triethylamine and diisopropylamine.

32.-34. (canceled)

35. A compound of formula (III) or formula (IV), or a salt thereof

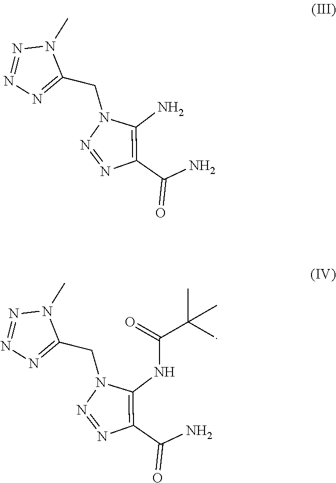

36.-37. (canceled)

38. The process according to claim 5 wherein the process is carried out in flow.

39. The process according to claim 5 wherein the process is carried out in batch.

40. The process according to claim 10 wherein the process is carried out in flow.
